SEQUENCE LISTING (16 sequences)

SEQ ID NO: 1 - DNA, 75 bp, synthetic construct
  source 1..75: mol_type = other DNA
aatgatacggcgaccaccgagatctacactctttccctacacgacgctcttccgatctggcctgctttgtttgcc

SEQ ID NO: 2 - DNA, 53 bp, synthetic construct
  source 1..53: mol_type = other DNA
gtgactggagttcagacgtgtgctcttccgatctcctccaccaccgctacctc

SEQ ID NO: 3 - DNA, 64 bp, synthetic construct
  source 1..64: mol_type = other DNA
caagcagaagacggcatacgagatcgtgatgtgactggagttcagacgtgtgctcttccgatct

SEQ ID NO: 4 - DNA, 64 bp, synthetic construct
  source 1..64: mol_type = other DNA
caagcagaagacggcatacgagatacatcggtgactggagttcagacgtgtgctcttccgatct

SEQ ID NO: 5 - DNA, 64 bp, synthetic construct
  source 1..64: mol_type = other DNA
caagcagaagacggcatacgagatgcctaagtgactggagttcagacgtgtgctcttccgatct

SEQ ID NO: 6 - DNA, 64 bp, synthetic construct
  source 1..64: mol_type = other DNA
caagcagaagacggcatacgagattggtcagtgactggagttcagacgtgtgctcttccgatct

SEQ ID NO: 7 - DNA, 64 bp, synthetic construct
  source 1..64: mol_type = other DNA
caagcagaagacggcatacgagatcactgtgtgactggagttcagacgtgtgctcttccgatct

SEQ ID NO: 8 - DNA, 64 bp, synthetic construct
  source 1..64: mol_type = other DNA
caagcagaagacggcatacgagatattggcgtgactggagttcagacgtgtgctcttccgatct

SEQ ID NO: 9 - DNA, 64 bp, synthetic construct
  source 1..64: mol_type = other DNA
caagcagaagacggcatacgagatgatctggtgactggagttcagacgtgtgctcttccgatct

SEQ ID NO: 10 - DNA, 64 bp, synthetic construct
  source 1..64: mol_type = other DNA
caagcagaagacggcatacgagattcaagtgtgactggagttcagacgtgtgctcttccgatct

SEQ ID NO: 11 - DNA, 64 bp, synthetic construct
  source 1..64: mol_type = other DNA
caagcagaagacggcatacgagatctgatcgtgactggagttcagacgtgtgctcttccgatct

SEQ ID NO: 12 - DNA, 64 bp, synthetic construct
  source 1..64: mol_type = other DNA
caagcagaagacggcatacgagataagctagtgactggagttcagacgtgtgctcttccgatct

SEQ ID NO: 13 - DNA, 64 bp, synthetic construct
  source 1..64: mol_type = other DNA
caagcagaagacggcatacgagatgtagccgtgactggagttcagacgtgtgctcttccgatct

SEQ ID NO: 14 - DNA, 64 bp, synthetic construct
  source 1..64: mol_type = other DNA
caagcagaagacggcatacgagattacaaggtgactggagttcagacgtgtgctcttccgatct

SEQ ID NO: 15 - DNA, 64 bp, synthetic construct
  source 1..64: mol_type = other DNA
caagcagaagacggcatacgagatttgactgtgactggagttcagacgtgtgctcttccgatct

SEQ ID NO: 16 - DNA, 64 bp, synthetic construct
  source 1..64: mol_type = other DNA
caagcagaagacggcatacgagatggaactgtgactggagttcagacgtgtgctcttccgatct

(19) United States
(12) Patent Application Publication
(10) Pub. No.: US 2024/0151731 A1
(43) Pub. Date: May 9, 2024

(54) MEMBRANE PROTEIN INTERACTION SCREENING PLATFORM BASED ON CELL-CELL ADHESION EFFECTS

(71) Applicant: SUZHOU INSTITUTE OF SYSTEMS MEDICINE, Suzhou, Jiangsu (CN)

(72) Inventors: Guideng LI, Suzhou (CN); Yuqian WANG, Suzhou (CN); Juan YANG, Suzhou (CN); Zhe WANG, Suzhou (CN)

(73) Assignee: SUZHOU INSTITUTE OF SYSTEMS MEDICINE, Suzhou, Jiangsu (CN)

(21) Appl. No.: 18/402,461

(22) Filed: Jan. 2, 2024

Related U.S. Application Data
(63) Continuation of application No. PCT/CN2022/102875, filed on Jun. 30, 2022.

(30) Foreign Application Priority Data
Jul. 1, 2021 (CN) 202110743776.2

(51) Int. Cl. G01N 33/68; C12N 15/10

(52) U.S. Cl. CPC G01N 33/6845; C12N 15/1096

(57) ABSTRACT

The present application relates to the field of cell immunity, and discloses an interacting protein screening platform for preparing a cell membrane. A cell membrane protein and a membrane protein encoding gene to be tested are respectively expressed in different cells; after staining, cells expressing different membrane proteins and cells expressing target proteins are co-incubated; and then interacting membrane proteins are obtained by screening, and specific sequences thereof are determined by sequencing. The screening platform provided in the present application can screen a variety of cell membrane proteins and membrane protein-based engineering cells, and has strong specificity, thereby greatly improving the efficiency of screening interacting proteins and reducing the difficulty, providing a simple and convenient tool for studying the mechanism of action of cell membrane proteins, and improving the screening throughput.

### CROSS REFERENCE TO RELATED APPLICATIONS

The present application is a Continuation Application of International Application No. PCT/CN2022/102875, filed on Jun. 30, 2022, which claims priority to the Chinese Patent Application No. CN202110743776.2, filed on Jul. 1, 2021 to the China National Intellectual Property Administration, and entitled “Membrane Protein Interaction Screening Platform Based on Cell-Cell Adhesion Effects”, the entire contents of each of which are incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 28, 2023, is named “Sequence_Listing_7166-0203PUS1.xml” and is 15,748 bytes in size. The sequence listing contained in this .XML file is part of the specification and is hereby incorporated by reference herein in its entirety.

### TECHNICAL FIELD

The present application relates to the field of immunology-related protein screening, and in particular, to a cell membrane surface protein interaction screening platform for screening specific binding proteins.

### BACKGROUND

Proteins serve as the primary executors of cellular function, and membrane proteins account for more than 30% of the human proteome. Cellular communication is usually controlled by the membrane protein interaction, and a majority of the signal pathways are initiated by the activation of cell surface receptors. Therefore, the interaction between membrane proteins plays an irreplaceable role in the communication between cells and intracellular signal transduction. Accurate identification of interacting proteins that are involved in intercellular communication is extremely important for us to understand the mechanisms of extracellular signal transduction and facilitate the design of potential drug targets.

The existing techniques for studying protein-protein interactions are as follows:

1. Co-immunoprecipitation (Co-IP): the co-immunoprecipitation technique is a classical method for studying protein-protein interactions based on the specific antibodies against the proteins of interest. The basic principle of the method involves the selective capture of a target protein along with its interacting partners from a complex mixture using specific antibodies, and further analyze the protein using methods such as mass spectrometry or Western blot to obtain information on protein-protein interactions. The co-immunoprecipitation technique has a wide range of applications, including cell biology, molecular biology, and biochemistry. When the co-immunoprecipitation technique is combined with other methods such as mass spectrometry or Western blot, interacting protein complexes that bind in a native state can be obtained through separation.

2. pMHC multimer labeling technique: in recent years, the fluorescent molecule labeled pMHC multimer combined with flow cytometry has become one of the most important detection tools in immunoassay, and has been widely used in the identification and separation of antigen-specific T cells. The pMHC is a soluble oligomeric form of MHC molecules. Through exploiting the robust binding affinity between avidin and biotin, several biotinylated pMHC molecules bind to fluorescently labeled avidin, forming a multimer. The multimer exhibits enhanced binding capacity to T cells compared to the monomeric form, and the complex formed with antigen-specific T cells is more stable. Consequently, the antigen-specific T cells can be efficiently identified and screened using flow cytometry. In addition to fluorescently labeled pMHC multimers, other molecules such as DNA barcodes and heavy metal ions have also been developed to labele pMHC multimers. The pMHC multimer labeling technology, combined with microfluidics and single-cell sequencing technologies, not only enables high-throughput screening of antigen-specific T cells, but also allows for simultaneous obtain of the TCR information from these T cells.

3. Yeast two-hybrid system: the yeast two-hybrid system, based on the reconstitution of transcriptional activators, is a classical method for studying protein-protein interactions in cells. However, the detectable range of the method is limited to proteins in the nucleus. To address the need for detecting interactions between membrane proteins, the yeast two-hybrid system based on split ubiquitin recombination has been developed. Ubiquitin, acting as a degradation signal molecule, is divided into N-terminal and C-terminal portions, each connected to a protein of interest. When interaction occurs between the two proteins, the split-ubiquitin molecules can recombine. The complementary reconstituted intact ubiquitin molecule can be recognized by ubiquitin-specific proteases, leading to the cleavage of the transcription factor attached to the C-terminus of ubiquitin. This cleavage allows the transcription factor to enter the cell nucleus and activate the transcription expression of reporter genes. The presence or absence of reporter gene expression can be used to determine whether there is an interaction between membrane proteins. The yeast two-hybrid system based on split ubiquitin recombination can be used not only for large-scale detection of interactions between membrane proteins but also for studying the interaction between antigens and antibodies.

4. Fluorescence resonance energy transfer (FRET): fluorescence resonance energy transfer is an energy transfer phenomenon between two fluorophores. If the excitation spectrum of one fluorophore coincides with the emission spectrum of another fluorophore, when the former fluorophore is excited, the energy can be transferred non-radiatively by dipole-dipole coupling and excite the latter fluorophore. Two fluorophores with this property are separately constructed into proteins to be tested, and whether there is an interaction between the two proteins can be known through the excitation of the fluorophores. The FRET method has high sensitivity and can intuitively provide location and quantitative information of protein-protein interactions, providing convenience for real-time dynamic study of protein-protein interactions in living cells.

5. Proximity Labeling Techniques

(1) APEX:

APEX is a genetically engineered peroxidase used as a labeling enzyme in electron microscopy (TM) and live cell proteomics. APEX is genetically fused to a target protein and stably expressed in cells, the cells are fixed and covered with diaminobenzidine (DAB) and hydrogen peroxide solution. Local deposition formed by APEX-catalyzed DAB polymerization can be observed by TM. When analyzing with proteomic profiling, biotin-phenol is added to the culture solution as a substrate. After treating the living cells with hydrogen peroxide for 1 minute, APEX catalyzes the oxidation of biotin-phenol, thereby generating a biotin-phenoxy radical that can covalently bind to an endogenous protein interacting with the APEX-target protein. The bound protein is enriched using streptavidin beads and identified by mass spectrometry to obtain information on the protein interacting with the target protein.

(2) BioID

The basic principle of BioID involves fusing a biotin ligase and bait protein in target cells. After adding biotin, the protein that interacts with or close to the bait protein will be labeled with biotin by biotin ligase. Subsequently, biotinylated proteins are purified using streptavidin affinity and subjected to mass spectrometry for identification. The experimental results are then compared to the control group to obtain the protein interacting with the bait protein. The experimental procedure includes 4 main steps: constructing a fusion expression vector for biotin ligase and the bait protein, stably expressing the fusion protein in the target cells, adding an appropriate amount of biotin, purifying the biotinylated protein by streptavidin and magnetic beads followed by mass spectrometry identification. The interacting proteins identified by BioID are naturally interacting with the bait protein in cells, and weakly bound proteins and transient interacting proteins can also be detected.

(3) PUP-IT

PUP-IT utilizes the Pup ligase PafA in bacteria to capture protein-protein interactions by labeling adjacent proteins with a small protein Pup. The bacterial Pup protein tagging system genetically fuses pafA (a gene encoding Pup ligase) and a bait protein. In the presence of ATP, pafA catalyzes the phosphorylation of Glu at the C-terminus of the Pup, and then the Glu at the C-terminus of pup is coupled to a lysine residue chain of a target protein. At present, PUP-IT has been applied to the study of T cell surface receptor CD28, and several CD28-interacting proteins have been identified. Meanwhile, a large number of potential new CD28-interacting proteins have also been discovered.

6. Trogocytosis:

Trogocytosis is a unique membrane transfer phenomenon between T lymphocytes and antigen-presenting cells. T cells are contacted with antigen-presenting cells after specific recognition, and the membrane surface proteins in the contact area of the contacted cells are transferred to each other due to trogocytosis. Screening for the precise pairing of TCR-antigens is achieved by labeling T cells and the membrane surface protein transfer of target cells recognized by the T cells. The trogocytosis technique has a wide range of applications, which can be used to screen antigens recognized by TCR with any HLA type, and is also suitable for screening antigens presented by MHCI or MHCII, thereby being used to identify potential antigens relevant to other immunotherapies in addition of tumors, such as autoimmune disease antigens. The trogocytosis technique also has high sensitivity and can simultaneously screen 10⁴-10⁵ antigenic epitopes.

7. T-Scan:

T-Scan utilizes the physiological process of a T cell killing function, in which granzyme B (GzB) is transported into target cells after the TCR on T cell surface is recognized by the target cells. The activity of GzB in the target cells is detected by the activation of a fluorescent reporter gene, thereby screening out target cells carrying antigen peptide information specifically recognized by TCR. A cleavable sequence that can be specifically recognized by GzB is constructed into a fluorescent protein and expressed in antigen-presenting cells. Upon the antigen-presenting cells specifically recognize the T cells, the T cells release GzB, which enters the antigen-presenting cells and cleaves the cleavable sequence, restoring the integrity of the fluorescent protein. The antigen-presenting cells expressing fluorescence are isolated by flow sorting and sequenced to obtain antigen sequences specifically recognized by TCRs. As a high-throughput, genome-wide technical platform, T-Scan can systematically screen antigens specifically recognized by TCRs.

8. Signaling and antigen-presenting bifunctional receptors (SABRs):

The SABRs method utilizes engineered pMHC complexes to initiate activation signals. SABRs are formed by the fusion of the extracellular pMHC complex and the intracellular CD28-CD3 signal domains. Upon SABRs-antigen-presenting cells and paired T cells recognize each other, the NFAT signal downstream of the antigen-presenting cells will be activated, thereby inducing the expression of a fluorescent protein. These cells expressing fluorescence can be sorted by flow cytometry, and the antigen information carried by these cells can be obtained after next-generation sequencing, thereby obtaining the accurate pairing information between TCR and antigens. The SABR technique is suitable for screening antigens recognized by TCR of any HLA type.

The above techniques still have the following challenges:
1. Co-immunoprecipitation:
\textcircled{1} transient protein interactions may be missed; \textcircled{2} the false positive rate is high, mainly because the detected binding may be caused by the action of a third substance rather than direct protein-protein interactions; \textcircled{3} only specific interactions between certain proteins can be detected in a single experiment; \textcircled{4} the operation process is complicated.
2. pMHC multimer labeling technique:
\textcircled{1} limited availability of fluorescent dyes restricts the detectable range in a single experiment; \textcircled{2} the preparation of pMHC tetramer is complicated and expensive.
3. Yeast two-hybrid system:
some proteins have regions with low affinity for other proteins on their surface, which are prone to form proteosome complexes, causing reporter gene expression and resulting in false positive results.
4. Fluorescence resonance energy transfer: low throughput and requires a distance of less than 100 angstroms between two fluorophores.
5. Proximity labeling techniques
(1) APEX2: requires the addition of hydrogen peroxide for the reaction to occur, potentially causing cell damage or oxidative stress when applied to live cells.
(2) BioID: \textcircled{1} it cannot distinguish whether identified proteins directly or indirectly interact with the bait protein; \textcircled{2} proteins without accessible amine groups cannot be biotinylated, making detection impossible.
6. Trogocytosis: validated only in TCR-antigen screening, and it needs to be further explored whether it can be applied to other membrane protein interactions.
7. T-Scan: \textcircled{1} screened antigens must be encodable; \textcircled{2} the technique is not suitable for non-peptide antigens.
8. Signaling and antigen-presentation bifunctional receptors: at present, they can only be used to screen the antigen peptides recognized by a single TCR at a time, and the number of antigens expressed in the antigen library is limited to 10⁶.

Compared to protein-protein interactions in the cytoplasm and nucleus, detecting protein-protein interactions on cell membranes faces greater challenges. The main reasons include: membrane proteins are not easy to extract; the interaction time is short and the interaction force is weak; the traditional methods for studying protein-protein interactions are not completely suitable for membrane proteins; and the mechanism of protein-protein interactions on cell membranes remains unclear.

### SUMMARY OF THE INVENTION

To identify and screen for potential interacting proteins of cell membrane proteins, the present application provides a membrane protein interaction detecting platform based on cell doublets(a pair of two cells) formation derived from specific adhesion effects in living cells. After co-culturing cells expressing different membrane proteins, specific doublets will occur between cells when interacting membrane proteins specifically recognize each other. After screening adherent cells, gene sequences of the interacting membrane proteins can be obtained by sequencing and other methods.

The platform is simple, broad-spectrum, and efficient, which can not only identify the interaction between membrane proteins, but also screen the membrane protein pairs that interact with each other with high throughput. In addition, the platform may be used to separate/screen specific target cells and assess the titer of a specific antibody, such as a bispecific antibody, etc.

In the first aspect, the present application provides a membrane protein interaction detecting platform, comprising: (1) co-culturing cells expressing different proteins; (2) fixing and screening cell doublets; (3) obtaining sequences of interacting membrane proteins through performing sequence analysis.

In some embodiments, to enable expression of the membrane proteins on cell surfaces, the detection further includes introducing expression vectors containing nucleic acid molecules encoding the membrane proteins into cells for expression. The methods of introducing the expression vectors into cells are performed by any method that enables cells to express the membrane proteins, including but not limited to viral vector infection, electroporation, and liposome method.

In some embodiments, the expression vectors include vectors capable of expression in eukaryotic cells, such as viral expression vectors and eukaryotic expression vectors, wherein the viral expression vectors include, but are not limited to, retroviral expression vectors, lentiviral expression vectors, adenoviral expression vectors, and adeno-associated viral expression vectors; the eukaryotic expression vectors include, but are not limited to, non-viral vectors such as pCMV and pEGF.

In some specific embodiments, the expression vector is retroviral; preferably, an MSGV retroviral; more preferably, the retroviral also includes packaging plasmids thereof, such as pRD114 and pHIT60.

In some specific embodiments, the expression vector is a lentiviral vector; preferably, a pCCLc lentiviral vector; more preferably, the lentiviral vector also includes packaging plasmids thereof, such as psPAX2 and pMD2.G.

In some embodiments, the expression vector includes genes encoding proteins and gene sequences directing membrane localization and membrane anchoring, and nucleic acids encoding different proteins are operably linked together and can be expressed on the surface of cell membranes.

In some embodiments, the membrane protein includes wild-type transmembrane proteins with transmembrane sequences or genetically engineered proteins or polypeptides fused to express one or more transmembrane sequences. Preferably, the transmembrane sequences include all transmembrane proteins or polypeptides with membrane localization functions, including but not limited to CD8 transmembrane sequences, CD28 transmembrane sequences, GPI anchor proteins, type A kinase anchor proteins, and all transmembrane protein sequences or polypeptides.

More preferably, the above membrane proteins include, but are not limited to, transmembrane glycoproteins, G protein-coupled receptors, immunoglobulins, viral proteins, antigen recognition receptors, antibodies, antigenic determinants, cytokine receptors, low-density lipoprotein receptors, and any engineered transmembrane proteins or polypeptides.

Further preferably, the transmembrane glycoproteins include, but are not limited to, CD40, CD40L, epidermal growth factor receptors, etc.; the G protein-coupled receptors include, but are not limited to, CD185 (CXCR5), chemokine receptor families, etc.; the immunoglobulins include, but are not limited to, CD80, CD86, PD-1, ICAM-1, CD19, etc.; the antigen recognition receptors include, but are not limited to, T cell receptors, B cell receptors, chimeric antigen receptors, etc. The cytokine receptors include, but are not limited to, type I cytokine receptors and type II cytokine receptors.

In some embodiments, the cells used in the construction of the detecting platform may be eukaryotic or prokaryotic cells. The eukaryotic cells include, but are not limited to, mammalian cells, yeast cells, insect cells, nematode cells, plant cells, or fungal cells. The mammalian cells include, but are not limited to, 293T, K562, Jurkat, Raji, EL4, etc.

In some specific embodiments of the present application, at least two identical or different cells are included in the screening platform for expressing different membrane proteins, wherein one/group of cells is used to express a known membrane protein and the other/group of cells is used to express a membrane protein to be tested which may interact with the known membrane protein. The cells may be eukaryotic cells or prokaryotic cells. The eukaryotic cells include, but are not limited to, mammalian cells, yeast cells, insect cells, nematode cells, plant cells, or fungal cells. The mammalian cells include, but are not limited to, 293T, K562, Jurkat, Raji, EL4, etc.

In some embodiments, fixatives are used to fix the cell doublets. The fixatives include, but are not limited to, all liquids that may maintain the cell structure, such as 4% paraformaldehyde fixative, 70% ethanol fixative, glutaraldehyde fixative, ethanol-formalin fixative, Carnoy fixative, etc.

In some embodiments, the detecting platform further includes labeling the cells expressing the membrane proteins with a tracer marker. The tracer markers include, but are not limited to protein tracer markers, biotin markers, fluorescent dye markers, magnetic bead markers, enzymatic markers, etc. The protein tracer markers are selected from green fluorescent protein, red fluorescent protein, yellow fluorescent protein, blue fluorescent protein, and all the proteins or polypeptides which may be labeled with conjugated antibodies marker, wherein the proteins labeled with the conjugated antibodies include, but are not limited to, all the proteins or polypeptides which may be recognized by the antibodies or ligands, such as NGFR, CD8, CD80, CD4, CAR, etc.

Preferably, the cells are traced and labeled with fluorescent dyes. The fluorescent dyes include, but are not limited to, dyes that enable the cells to carry fluorescent signals, such as live cell tracers, etc., which can be selected from CMFDA, Violet, Far-red, and other live cell tracers.

In a specific example, cells expressing different membrane proteins are separately labeled with different dyes.

In some embodiments, the interaction between membrane proteins is identified by the expression of reporter genes; preferably, the reporter genes are used to identify T cells activated by target antigens through the TCR-antigen interaction.

In some embodiments, the reporter gene is a promoter activity reporter gene, including but not limited to, NF-κB, NFAT, and AP-1; preferably, the reporter gene is an NFAT promoter reporter gene.

In some specific embodiments, the NFAT promoter-reporter gene is constructed into a vector and introduced into TCR-expressing cells; preferably, the vector is a retroviral vector; more preferably, the vector is introduced into the TCR-expressing cells by viral infection.

In some embodiments, the methods for screening the cell doublets by the detecting platform include, but are not limited to, methods commonly used for cell screening and separation in the art, such as fluorescence-activated cell sorting (FACS), droplet microfluidic technique, etc.; preferably, the screening is performed by FACS.

In some embodiments, the detecting platform sequence analysis is performed by sequencing technique. A variety of sequencing methods may be used to determine sequences of the interacting membrane proteins (e.g. antigen peptides or TCR), such as sequencing a polynucleotide encoding a polypeptide of interest. The polynucleotide sequencing method includes, but is not limited to, Sanger sequencing and next-generation sequencing (NGS). The NGS method includes, but is not limited to, a synthetic sequencing platform, an Illumina/Solexa platform (e.g. HiSeq and MiSeq), a 454 pyrosequencing platform (Roche), and a SOLiD platform (Applied BioSystems).

The polynucleotide sequences may be purified before polynucleotide sequencing. Polynucleotides may be purified using a technique widely used in scientific research, such as phenol-chloroform extraction, agarose gel separation, silica gel column-based purification, cesium chloride, similar purification methods, or combinations thereof. A particular purification method may be selected based on the particular polynucleotide to be purified. The polynucleotide sequences of interest may also be purified and further processed prior to sequencing, such as amplification by PCR method, barcoding, modification with sequencing adapters, reverse transcription, or any other processes prepared for sequencing.

In some embodiments, to screen for different membrane proteins that interact with known target antigen membrane proteins, the detecting platform expression vector described in the present application further includes any library expressing the membrane proteins, preferably a cDNA library expressing the membrane proteins to be tested. The library sequences described in the present application may refer to genome sequences published by GenBank, including but not limited to humans, mice, rats, chickens, and other animals. The antigen membrane proteins may be derived from one or more autologous and non-autologous proteins. Non-limiting examples include parasitic proteins, viral proteins, bacterial proteins, parasitic antigens, viral antigens, bacterial antigens, autoimmune disease-inducing proteins, autoantigens, tumor proteins, tumor antigens, cancer proteins, cancer antigens, exogenous antigens, endogenous antigens, neoantigens, toxins, etc.

In some specific embodiments, the cDNA library of the membrane proteins to be tested is selected from a HLA-A2-SCT cDNA library, wherein SCT is a three-part combination of the antigen peptide, β-microglobulin and HLA-A2 domain linked via GS linkers, and a disulfide bond modification is constructed into the GS linkers and HLA-A2. Preferably, the library includes about 12,000 A2 epitopes, and a neoantigen SCT cDNA library includes about 3,000 neoantigen epitopes.

In another specific embodiment, the cDNA library of the membrane proteins to be tested is selected from an A2-restricted SCT cDNA library; preferably, the library includes 12,055 public antigen peptide sequences from the Immune Epitope Database (IEDB).

In some embodiments, to cause effective cell adhesion, the ratio of the cells expressing the membrane proteins is from about 20000:1 to about 1:20000, preferably from 5:1 to 1:10000. In some embodiments, the ratio of the cells is about 5:1, 1:1, 1:5, 1:10, 1:20, 1:50, 1:100, 1:200, 1:500, 1:1000, 1:3000, 1:5000, 1:10000, or a ratio within a range defined by any two of the above ratios.

In some embodiments, the ratio of the cells is 5:1, 1:1, 1:5, 1:20, 1:3000, 1:5000, and 1:10000; preferably, the ratio is 5:1, 1:1, and 1:5.

In some embodiments, the cells undergoing cell adhesion include at least 5% of the total cells. In some embodiments, the cell doublets formation efficiency is about 60% or a value within a range defined by any two of the above values.

In a second aspect, the present application provides a method for identifying a cell membrane protein interaction using the detecting platform described in the first aspect.

In some embodiments, the interaction between cell membrane proteins can be, but is not limited to, binding between ligands and receptors, specific binding between TCR and antigens, specific binding between CAR and antigens, binding between viral proteins and receptors thereof, etc.

In some embodiments, the detecting platform includes the following steps: expressing a nucleic acid sequence fragment encoding a protein in cell A by genetic engineering means, and expressing the encoded protein in the cells, and the protein is any protein that can be anchored on the cell membranes;
co-incubating cells A with another cells B carrying identical or different membrane proteins, resulting in contact;
fixing the cells to maintain and stabilize the state of interaction between the cells; detecting or sorting for interacting cells that may adhere and aggregate, i.e. cell adhesion; identifying the membrane protein information carried by the adherent cells to obtain information on the interaction or expression level between the transmembrane proteins.

In some embodiments, the above membrane proteins include, but are not limited to, transmembrane glycoproteins, G protein-coupled receptors, immunoglobulins, viral proteins, antigen recognition receptors, antibodies, antigenic determinants, cytokine receptors, low-density lipoprotein receptors, and any modified transmembrane proteins or polypeptides.

The transmembrane glycoproteins include, but are not limited to, CD40, CD40L, epidermal growth factor receptors, etc.; the G protein-coupled receptors include, but are not limited to, CD185 (CXCR5), chemokine receptor families, etc.; the immunoglobulins include, but are not limited to, CD28, CD80, CD86, PD-1, ICAM-1, CD19, etc.; the antigen recognition receptors include, but are not limited to, T cell receptors, B cell receptors, chimeric antigen receptors, etc.

The cytokine receptors include, but are not limited to, type I cytokine receptors and type II cytokine receptors. The viral proteins include, but are not limited to, COVID-19 spike protein, HBs, etc.

In some embodiments, the detectable tracer marker used to identify the above cell doublets includes, but is not limited to, all live cell labeling methods that can distinguish cells A from cells B, such as protein tracer markers, biotin markers, fluorescent dye markers, magnetic bead markers, and enzymatic markers.

Preferably, the fluorescent dye markers include, but are not limited to, dyes that enable the cells to carry fluorescent signals, such as live cell tracers, which can be selected from CMFDA, Violet, Far-red, and other live cell tracers.

In some embodiments, the fixative used to fix the adhered cells includes, but is not limited to, all liquids that may maintain the cell structure, such as 4% paraformaldehyde fixative, 70% ethanol fixative, glutaraldehyde fixative, ethanol-formalin fixative, Carnoy fixative, etc.

In some embodiments, the sorting method includes, but is not limited to, all methods that can sort out adherent cells, such as fluorescence-activated cell sorting (FACS), droplet microfluidic technique, etc.

In some embodiments, the detecting step includes: extracting polynucleotides from the sorted adherent cells; performing PCR amplification using the oligonucleotide barcode-labeled primers; and after purifying the amplification products, further obtaining information on the membrane proteins interacting with known proteins using a nucleotide sequencing technique.

Preferably, the oligonucleotide barcode may be directly or indirectly conjugated to the polynucleotides extracted after screening the adherent cells.

More preferably, the polynucleotides are selected from genomic DNA, vector DNA, plasmid DNA, mRNA, DNA amplified by PCR, and cDNA produced by reverse transcription, etc.

Further preferably, the nucleotide sequencing technique includes, but is not limited to, any method by which nucleotide sequence information can be obtained, such as Sanger sequencing and next-generation sequencing (NGS).

In a third aspect, the present application provides a method for separating and screening target cells using the platform described in the first aspect, comprising the following steps: co-culturing the cells A expressing a specific membrane protein with target cells B to be separated and screened, the target cells B expressing proteins that can interact with the specific membrane protein, resulting in the adhesion between the cells A and B, and then fixing, separating and screening the target cells B.

In some embodiments, the target cells include, but are not limited to, target cells that specifically bind to TCR, target cells that specifically bind to CAR, and cells that specifically bind to target proteins; In some embodiments, the target cells are any cells that may be recognized by cells expressing antigen-specific TCR or cells expressing binding molecules. The target cells may be primary cells, ex vivo cultured cells, tumor-infiltrating cells, autoimmune cells, pathogen-infected cells, cancer or tumor-derived cells, and immortalized cell lines. The immortalized cell lines include, but are not limited to, K562 cells, HEK293 cells and derivatives thereof (e.g. HEK 293T cells), 3T3 cells and derivatives thereof, Chinese hamster ovary (CHO) cells and derivatives thereof, and HeLa cells and derivatives thereof.

In some embodiments, the target cells include cells expressing CD19, CD20, PD1, PDL1, CD3, CD40, CD28, CD86, and particular antigens; the target cells may also be selected from B cells, CD4 T cells, CD8 T cells, γ-δT cells, NKT cells, artificially constructed CART cells, TCR-expressing cells, and cancer cells expressing tumor-associated antigens.

In a fourth aspect, the present application provides a method for screening interacting proteins from membrane proteins using the platform described in the first aspect, which has one or more of the following uses:
(1) screening T cell receptors (TCR) and target antigens thereof;
(2) screening chimeric antigen receptors (CAR) and target antigens thereof;
(3) screening antigens and antibodies or receptors thereof;
(4) screening cytokines and receptors thereof.

In some embodiments, the membrane proteins include, but are not limited to, wild-type membrane proteins, any proteins overexpressed by genetic engineering methods that can be localized to the membranes, or transmembrane protein library.

In some embodiments, the antigens, which can be classified as endogenous antigens and exogenous antigens depending on the location of processing in the cells, may be presented to the surface of antigen-presenting cells via an MHC molecule.

The endogenous antigens refer to newly synthesized antigens in the antigen-presenting cells, including but not limited to viral antigens and tumor antigens, which are presented to CD8⁺ T cells as an antigen peptide-major histocompatibility complex class I molecule (MHC I) complex.

The exogenous antigens refer to that the antigen-presenting cells are taken up from the outside, and presented to the CD4⁺T cell in the form of an antigen peptide-major histocompatibility complex class II molecule (MHC II) complex after degrading into short peptides.

The MHC includes MHC I, MHC II, and MHC III genes, which respectively encode MHC I, MHC II, and MHC III molecules. Its sources includes, but are not limited to, human MHC (HLA complex), mouse MHC (H-2 complex), rat MHC (H-1 complex), etc.

Preferably, the MHC includes alleles at HLA-A, B, C, E, F, G, H, J, K, L, and other loci, which encode class I antigens such as HLA-A antigen, B antigen, C antigen, E antigen, F antigen, G antigen, H antigen, J antigen, K antigen, and L antigen.

Preferably, a gene region of the MHC II molecules includes HLA-DP, DQ, DR, DN, DO, and DM. In some embodiments, the antigens may be divided into natural antigens, synthetic antigens, and genetically engineered antigens according to the preparation method. The synthetic antigens refer to artificially synthesized polypeptides with antigenic properties. The genetically engineered antigens refer to antigens that express genes encoding immunogenic amino acid sequences in an eukaryotic or prokaryotic expression vector, preferably including but not limited to a class of antigens or a class of antigen library expressed in cells in the form of single-chain trimer (SCT). The SCT refers to a three-part combination of the antigen peptide, β-microglobulin and MHC domain linked via GS linkers, and a disulfide bond modification is constructed into the GS linkers and MHC.

In some embodiments, the antigens are selected from a HLA-A2-SCT cDNA library, preferably comprising about 12,000 A2 epitopes and about 3,000 novel epitopes.

In some embodiments, the antigens are selected from an A2-restricted SCT cDNA library, wherein the library includes 12,055 public antigen peptide sequences from the Immune Epitope Database (IEDB).

In some specific embodiments, the antigen peptide is selected from NYESO and MART1.

In some embodiments, the TCR includes, but is not limited to, human-derived and murine-derived endogenous and exogenous TCR.

Preferably, the endogenous TCR includes, but is not limited to, TCR derived from a population of T cells such as primary T cells, killer T cells, and memory T cells.

Preferably, the exogenous TCR includes TCR alpha and beta chains. Polynucleotide sequences encoding at least one TCR are ligated into the same plasmid vector.

In some specific embodiments, the TCR is selected from F5 TCR, 1G4 TCR, and Neo TCR.

In a fifth aspect, the present application provides a method for assessing the titer of a multispecific antibody using the platform described in the first aspect, which includes the following steps: labeling cells expressing specific proteins on cell membranes, co-culturing the labeled cells with the multispecific antibodies, fixing and detecting the proportion of labeled adherent cell to assess the titer of the multispecific antibodies.

In some embodiments, cells expressing different kinds of proteins are labeled with different markers, preferably, using fluorescent tracers for labeling; more preferably, the fluorescent tracers are selected from Violet, CMFDA, etc.

In some embodiments, the proportion of the labeled adherent cell populations is detected using FACS; preferably, the titer is assessed by the proportion of the labeled adherent cell populations.

In a specific embodiment, the multispecific antibody is bispecific, and the cells are separately labeled with two different fluorescent tracers; preferably, the specific proteins expressed by the cells are selected from CD3 and CD19.

In a sixth aspect of the present application, the detecting platform further comprises the following steps:
(1) co-culturing: mixing cells expressing different membrane proteins at an appropriate ratio, incubating under certain conditions;
(2) flow cytometry analyzing and sorting: fixing the cells after incubation to keep interacting cells in an adherent state, and sorting cells with both types of fluorescence simultaneously by a cell flow cytometry;
(3) sequencing and analyzing: amplifying screened cellular DNA by polymerase chain reaction (PCR) technology, and then performing next-generation sequencing to obtain membrane protein sequences with specifically adhere to known membrane proteins.

Preferably, before the co-cultivation, the method further includes the following steps: constructing fusion plasmid: constructing target protein sequences into a plasmid vector; stably expressing protein: packaging target proteins into a virus by transfection, and expressing the target proteins on specific cells by infection to obtain a cell line stably expressing the membrane proteins;
live cell tracking labeling: labeling the cells with live cell tracking dyes.

In some embodiments, the live cell tracking labeling in the method includes cell staining and fluorescent protein labeling. The cell staining is a staining agent commonly used in fluorescence-activated cell sorting (FACS), including CMFDA, Violet, DAPI, etc., which may also be used in cell labeling by expressing various fluorescent proteins in cells.

In some embodiments, to produce effective cell adhesion, the ratio of the cells expressing the membrane proteins is from about 20000:1 to about 1:20000, preferably from about 5:1 to about 1:10000. In some embodiments, the ratio of the cells is about 5:1, 1:1, 1:5, 1:10, 1:20, 1:50, 1:100, 1:200, 1:500, 1:1000, 1:3000, 1:5000, 1:10000, or a ratio within a range defined by any two of the above ratios. In some embodiments, the ratio of the cells is 5:1, 1:1, 1:5, 1:20, 1:3000, 1:5000, 1:10000; preferably, the ratio is 5:1, 1:1, 1:5.

### Definitions

Membrane proteins: the term “membrane proteins” used in the present application refers to protein components on cell membranes, including but not limited to membrane surface proteins and transmembrane proteins. The membrane proteins may be expressed by the cell line itself or may be overexpressed by engineering.

Cell doublets: the term “cell doublets” used in the present application refers to a specific pair of cells or two specific cells generated by cell adhesion mediated by membrane protein interactions between different cells.

Target cells: the term “target cells” used in the present application refers to any cells that may be recognized by cells expressing a known protein. The target cells include, but are not limited to, immortalized cell lines, and may also be primary cells, tumor-derived cells, etc., wherein the immortalized cells include, but are not limited to, K562 cells, Raji cells, and engineered cell lines as described above. In the example of identifying TCR epitopes, the target cells may be any cells expressing antigens and MHC molecules capable of presenting the antigens.

Co-culture: the term “co-culture” used in the present application refers to the incubation of two or more cells at a suitable ratio in a particular buffer at 37° C. for a period of time, allowing the cells to contact, recognize, and specifically interact with each other. Co-culture involves culturing the cells together under conditions sufficient for cell adhesion to occur. Cell culture vessels include, but are not limited to, 1.5 ml EP tubes and 96 well U-bottom plates.

Separation: the term “separation” refers to separating cells, cell types, or cell populations of interest from other cell populations, and generally includes enrichment of a cell of interest relative to other cells. Any of the cells described herein may be separated, such as target cells or TCR-expressing cells.

Appropriate ratio: the term “appropriate ratio” used in the present application refers to the ratio of cells at which the highest double-positive ratio occurs between paired cells, as determined by a pre-experiment.

“Nuclear factor of activated T cells” (NFAT) is a family of transcription factors that have been shown to be important in immune responses. One or more members of the NFAT family are expressed in most cells of the immune system, and NFAT is also involved in the development of the heart, skeletal muscle, and nervous system. NFAT was originally discovered as an activator of IL-2 transcription in T cells, serving as a regulator of T cell immune responses, but was later discovered to play an important role in regulating many other body systems. NFAT transcription factors are involved in many physiological processes as well as in the development of various diseases, such as inflammatory bowel disease and various cancers. Under resting conditions, the NFAT transcription factors are fully phosphorylated and reside in the cytoplasm. In the case of T cell activation, the binding of TCR to the target peptide-MHC results in a local influx of calcium ions, thereby activating calcineurin (a calcium-dependent phosphatase). Dephosphorylation of NFAT by calcineurin then results in the translocation of NFAT into the nucleus and subsequent transcription of NFAT-controlled genes. Therefore, the activity of NFAT transcription factors may be used to detect the conduction of early TCR signaling.

The present application has the following beneficial effects.

1. The present application utilizes a cell transfection or cell infection method is used to enable the cells to carry a certain membrane protein or a certain group of a membrane protein library. After culturing the cells, flow cytometry or other technical means are used to separate the cell clusters that have undergone cell adhesion, and high-throughput sequencing is used to obtain the membrane protein information carried by the adherent cell clusters, thereby being used for screening proteins interacting with a certain target protein, which greatly improves the screening efficiency and has strong specificity.

2. The present application discloses a membrane protein interaction detecting platform, which is convenient, simple, efficient, and widely applicable, and can effectively detect the membrane protein interaction between cells.

3. The detecting platform described in the present application can be used to rapidly and efficiently identify and verify the interaction between membrane proteins, and can also be used to simply and conveniently screen target cells interacting with the membrane proteins, and can further be applied for screening and identifying TCR, CAR and target antigens thereof, as well as antigens, cytokines and receptors thereof, and has great development potential.

4. The detecting platform described in the present application can also be used for assessing the titer of multispecific antibodies.

### BRIEF DESCRIPTION OF THE DRAWINGS

To illustrate the technical solutions in examples of the present application more clearly, the drawings required for the examples will be briefly introduced below. The drawings described below are merely some examples recorded in the present application. For those skilled in the art, other technical solutions and corresponding drawings can also be obtained based on these drawings without creative labor.

FIG. 1 is a schematic diagram of a cell adhesion process.

FIG. 2 illustrates the verification of the interaction between CD40L and CD40 proteins by a cell adhesion method.

FIG. 3 illustrates the verification of the interaction between CD28 and CD80/86 proteins by a cell adhesion method.

FIG. 4 illustrates the identification of TCR-specific antigens by a cell adhesion method.

FIG. 5 illustrates the identification of CAR-specific antigens by a cell adhesion method.

FIG. 6 illustrates the identification of viral proteins and receptors thereof by a cell adhesion method.

FIG. 7 illustrates the separation of cells presenting specific TCR target antigens by a cell adhesion method.

FIG. 8 illustrates the specific separation of cells expressing target proteins by a cell adhesion method.

FIG. 9 illustrates the screening for TCR-specific antigens from a SCT library by a cell adhesion method.

FIG. 10 is a schematic diagram of a cell adhesion process combined with an NFAT promoter-reporter gene.

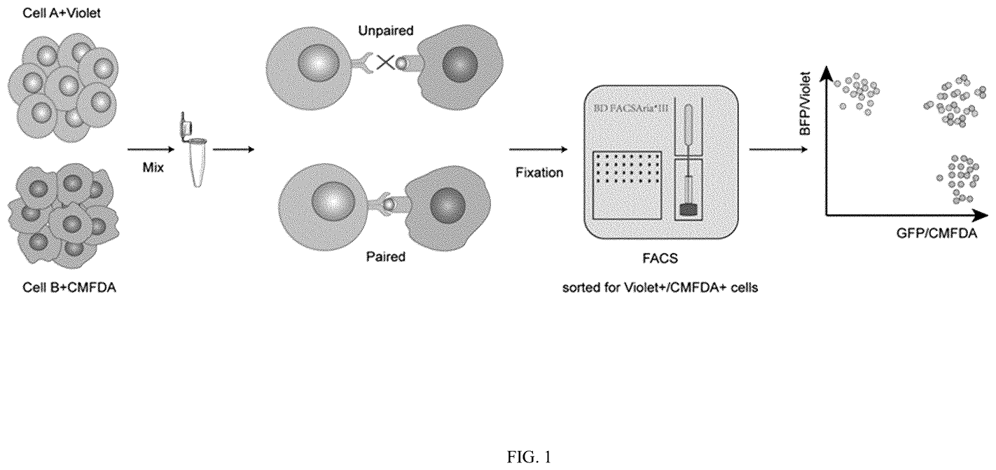

### DETAILED DESCRIPTION OF THE INVENTION

### Example 1. Preparation Method for Cell Membrane Protein Interaction Screening System

1. Plasmid Construction

Genes encoding CD40 (GenBank: M83312.1), CD40L(GenBank: X65453.2), CD28(GenBank: J02988.1), CD80(GenBank: BC042665.1), CD86(GenBank: KU284848.1) and CD8(GenBank: M12824.1/X13444.1) and other proteins, as well as F5 TCR, 1G4 TCR and Neo TCR genes carrying human or murine TCR constant regions, were separately constructed into an MSGV retroviral vector, wherein the MSGV vector is from the Eugene Barsov Laboratory, the restriction sequences of which have been modified to facilitate the cloning of the TCR genes, in the form of LNGFRΔ-P2A-TCRα-F2A-TCRβ. Genes encoding antigen peptide-MHC single chain trimer (SCT) and eGFP were constructed into a lentiviral vector. The vector backbone pHAGE6 is from Richard Mulligan Laboratories, the restriction sequences of which have been modified to facilitate the cloning of the SCT and MHC genes, wherein SCT is a three-part combination of the antigen peptide (NYESO, MART1), β-microglobulin and HLA-A2 domain linked via GS linkers, and a disulfide bond modification is constructed into the GS linkers and HLA-A2.

2. Cell Line Construction

The retroviral plasmid as described above and its packaging plasmids (pRD114 and pHIT60) or the lentiviral plasmid as described above and its packaging plasmids (psPAX2 and pMD2.G) were transfected into HEK-293T cells by using PEI transfection reagent. After 48 hours of transfection, the virus was filtered through a 0.45 μm filter and collected for infection. After the addition of 10 μg/mL polybrene to the collected virus supernatant, centrifugation was performed at 2500 rpm and 37° C. for 90 min to infect Jurkat, K562, or 293T cells. The above cells were purchased from the American Type Culture Collection (ATCC). After 48 hours of infection, TCR⁺CD8⁺Jurkat cells, eGFP⁺K562 cells, CD40⁺293T cells, CD40L+293T cells, CD28+ Jurkat cells, CD80⁺K562 cells and CD86⁺K562 cells were sorted by flow cytometry to obtain cell lines stably expressing specific proteins.

3. Live Cell Tracking Dye Labeling

TCR-Jurkat, CD28-Jurkat and CD40L-293T cells were resuspended at 2M/mL in PBS solution and incubated at 37° C. for 30 mins after the addition of Violet live cell tracking dye. SCT-K562, CD80/86-K562 and CD40-293T cells were resuspended at 2M/mL in PBS solution and incubated at 37° C. for 30 mins after the addition of CMFDA live cell tracking dye. The incubated cells were washed twice with PBS solution containing 2% FBS to wash out the dye remaining in the solution and be used for co-culture experiments.

4. Co-Culture and Flow Cytometry

Cells labeled with live cell tracking dye were added to a 1.5 mL EP tube at a cell ratio of Jurkat:K562=5:1 (total of 0.25M cells) (293T-CD40L:293T-CD40=5:1 for CD40L-CD40 group), mixed well, and then co-cultured at 37° C. for 30 mins. After co-culture, the cell mixture was fixed with 500 ul of a fixative. Fixed cells were analyzed by flow cytometry, and Violet+CMFDA+ cells were paired cells with cell adhesion due to interaction.

5. Library Construction

To be suitable for high-throughput and broad screening of interacting antigen proteins, an oligonucleotide library encoding candidate epitopes was synthesized by Twist Bioscience and used as a template for PCR amplification, and then inserted into a pCCLc lentiviral vector for co-expression with eGFP to construct a desired A2-restricted SCT cDNA library, which includes about 12,000 A2 epitopes and about 3,000 novel epitopes. The plasmid encoding the above antigen library and its packaging plasmids were transfected into 293T cells using a PEI transfection reagent. After 48 hours of transfection, the virus was collected and K562 cells were infected. After 48 hours, the expression of eGFP was detected, and if it reached more than 80%, it could be used for subsequent co-culture experiments.

6. Co-Culture with A2/Neo Library and Cell Sorting

In the co-incubation experiment of F5-Jurkat or 1G4-Jurkat cells with A2-SCT-K562 library cells and the co-incubation experiment of Neo-Jurkat cells with Neo-SCT-K562 library cells, 2M Jurkat cells were mixed with 2M K562 cells and co-cultured at 37° C. for 30 mins. After being fixed, the cell cultures were sorted by flow cytometry to obtain Violet+CMFDA⁺ cells.

7. PCR Amplification and Sequencing

The genomic DNA of the sorted cells was extracted using a DNA extraction kit and used as a template. Primer TruSeq-Univ-SCTfixed-F, primer TruSeq-Read2-SCTfixed-R and primer index (primer index is a short DNA fragment, which can be used to label the DNA sequence) were added for PCR amplification. Different samples were labeled with different index primers at the end of the DNA sequence. The primer sequences are shown in Table 1:

TABLE 1
Primer sequences

| | | |
|---|---|---|
| 1 | TruSeq-Univ-S | aatgatacggcgaccaccgagatctacactctttccctacacgacgctcttccgatctggcctgctttgtt |
| | CTfixed-F | tgcc |
| 2 | TruSeq-Read2- | gtgactggagttcagacgtgtgctcttccgatctcctccaccaccgctacctc |
| | SCTfixed-R | |
| 3 | Truseq-Adapter | caagcagaagacggcatacgagatcgtgatgtgactggagttcagacgtgtgctcttccgatct |
| | -Index-1 | |
| 4 | Truseq-Adapter | caagcagaagacggcatacgagatacatcggtgactggagttcagacgtgtgctcttccgatct |
| | -Index-2 | |
| 5 | Truseq-Adapter | caagcagaagacggcatacgagatgcctaagtgactggagttcagacgtgtgctcttccgatct |
| | -Index-3 | |
| 6 | Truseq-Adapter | caagcagaagacggcatacgagattggtcagtgactggagttcagacgtgtgctcttccgatct |
| | -Index-4 | |
| 7 | Truseq-Adapter | caagcagaagacggcatacgagatcactgtgtgactggagttcagacgtgtgctcttccgatct |
| | -Index-5 | |
| 8 | Truseq-Adapter | caagcagaagacggcatacgagatattggcgtgactggagttcagacgtgtgctcttccgatct |
| | -Index-6 | |
| 9 | Truseq-Adapter | caagcagaagacggcatacgagatgatctggtgactggagttcagacgtgtgctcttccgatct |
| | -Index-7 | |
| 10 | Truseq-Adapter | caagcagaagacggcatacgagattcaagtgtgactggagttcagacgtgtgctcttccgatct |
| | -Index-8 | |
| 11 | Truseq-Adapter | caagcagaagacggcatacgagatctgatcgtgactggagttcagacgtgtgctcttccgatct |
| | -Index-9 | |
| 12 | Truseq-Adapter | caagcagaagacggcatacgagataagctagtgactggagttcagacgtgtgctcttccgatct |
| | -Index-10 | |
| 13 | Truseq-Adapter | caagcagaagacggcatacgagatgtagccgtgactggagttcagacgtgtgctcttccgatct |
| | -Index-11 | |
| 14 | Truseq-Adapter | caagcagaagacggcatacgagattacaaggtgactggagttcagacgtgtgctcttccgatct |
| | -Index-12 | |
| 15 | Truseq-Adapter | caagcagaagacggcatacgagatttgactgtgactggagttcagacgtgtgctcttccgatct |
| | -Index-13 | |
| 16 | Truseq-Adapter | caagcagaagacggcatacgagatggaactgtgactggagttcagacgtgtgctcttccgatct |
| | -Index-14 | |

The first-step PCR product was purified using a PCR purification kit. The purified product was used as a template, and then the second PCR was performed using the primer TruSeq-Univ-SCTfixed-F and the corresponding primer index. After running the gel by agarose gel electrophoresis, the target fragment was cut and recovered, and samples were collected for next-generation sequencing.

### Example 2. Verification of Interaction Between CD40L and CD40 Proteins by a Cell Adhesion Screening Method

Cell lines stably expressing CD40 and CD40L proteins were first established. Genes encoding CD40 and CD40L proteins were constructed into an MSGV retroviral vector and transfected into 293T cells with a PEI transfection reagent for 48 hours and the virus was collected. 293T cells were infected with the virus for 48 hours and then sorted by flow cytometry to obtain cells highly expressing CD40 and CD40L.

Then, CD40L-293T cells were labeled with Violet live cell tracking dye, and CD40-293T cells were labeled with CMFDA live cell tracking dye. These cells were then co-cultured at a cell ratio of 5:1 at 37° C. for 30 mins and fixed with a fixative. Finally, the formation of Violet and CMFDA double-positive cell populations was observed and analyzed by confocal microscopy and flow cytometry.

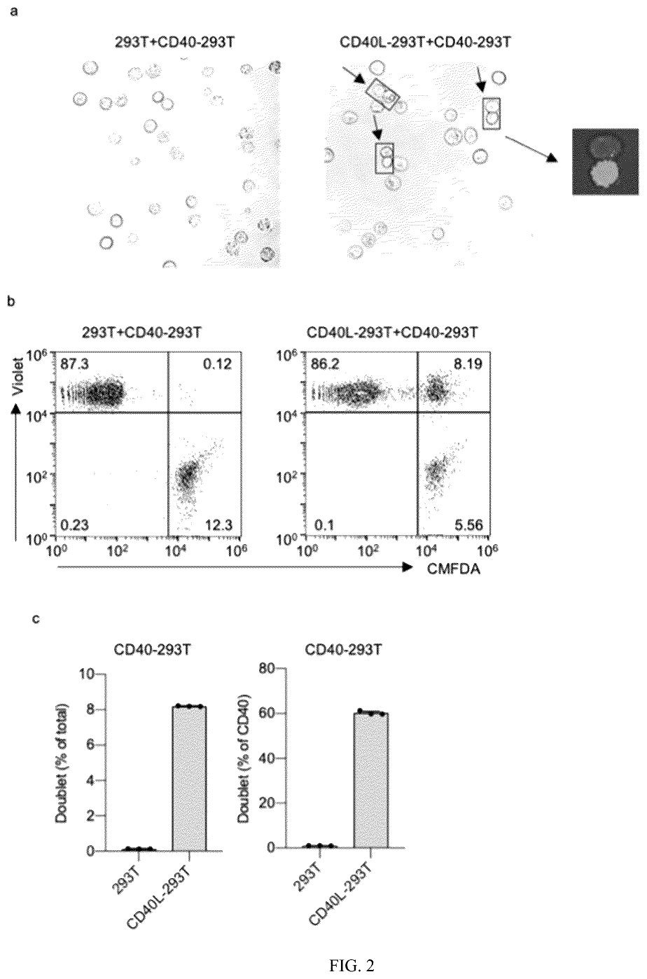

After co-culturing CD40L-293T cells (Violet+) and CD40-293T cells (CMFDA⁺) under the above conditions, the cell doublets could be observed by confocal microscopy. The cell doublets that express Violet and CMFDA respectively could be found after superposition of fluorescence. However, after co-culturing cells not expressing CD40L with CD40-293T cells, the overall cells showed an independent scattered state (FIG. 2a), demonstrating that the adherent cell pairs observed by confocal microscopy were caused by the mutual recognition of CD40L and CD40. Further analysis of the fixed cell co-cultures by flow cytometry showed that 8.19% of the total cells expressed both types of fluorescence in the pair group (FIG. 2b) and approximately 60% of the cells in the CD40-293T cell populations (CMFDA⁺) also expressed Violet fluorescence (FIG. 2c). The above results demonstrate that cell doublets forming can be used to identify the interaction between CD40-CD40L proteins.

### Example 3. Verification of Interaction Between CD28 and CD80/86 Proteins by a Cell Adhesion Method

Genes encoding CD28, CD80 and CD86 proteins were constructed into a pCCLc lentiviral vector and transfected into 293T cells with a PEI transfection reagent for 48 hours and the virus was collected. Jurkat cells were infected with CD28, and K562 cells were infected with CD80 and CD86, respectively. After 48 hours, these cells were sorted by flow cytometry to obtain cells highly expressing CD28, CD80 and CD86. CD28-Jurkat cells were labeled with Violet live cell tracking dye, and CD80-K562 and CD86-K562 cells were labeled with CMFDA live cell tracking dye. These cells were then co-cultured at a cell ratio of Jurkat:K562=1:5 at 37° C. for 30 mins and fixed with a fixative. The formation of Violet and CMFDA double-positive cell populations was analyzed by flow cytometry.

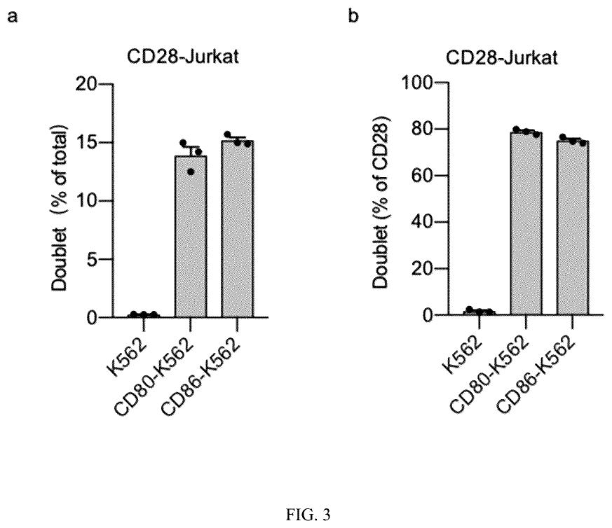

After co-culturing CD28-Jurkat cells (Violet⁺) with CD80-K562 and CD86-K562 cells (CMFDA⁺) respectively under the above conditions, further analyzed the fixed cell co-cultures by flow cytometry. About 15% of the total cells expressed both types of fluorescence in the pair group (FIG. 3a) and approximately 70% of the cells in the CD28-Jurkat (Violet⁺) populations also expressed CMFDA fluorescence (FIG. 3b). The above results demonstrate that cell doublets forming can be used to identify the interaction between CD28 and CD80/CD86 proteins.

### Example 4. Identification of TCR-Specific Antigens by a Cell Adhesion Method

Genes encoding F5 TCR, 1G4 TCR and huCD8 were constructed into an MSGV retroviral vector and transfected into 293T cells with a PEI transfection reagent for 48 hours and the virus was collected. Jurkat cells were infected with F5 TCR⁺ huCD8 and 1G4 TCR⁺huCD8 respectively for 48 hours and then sorted by flow cytometry to obtain cells highly expressing both TCR and CD8. Genes encoding NYESO-SCT and MART1-SCT were constructed into a pCCLc lentiviral vector and transfected into 293T cells with a PEI transfection reagent for 48 hours and the virus was collected. K562 cells were infected with the virus for 48 hours and then sorted by flow cytometry to obtain cells high expressing GFP. Jurkat cells overexpressing TCR were labeled with Violet live cell tracking dye, and K562 cells overexpressing SCT were labeled with CMFDA live cell tracking dye. These cells were then co-cultured at a cell ratio of 5:1 at 37° C. for 30 mins and fixed with a fixative. The formation of Violet and CMFDA double-positive cell populations was observed and analyzed by flow cytometry.

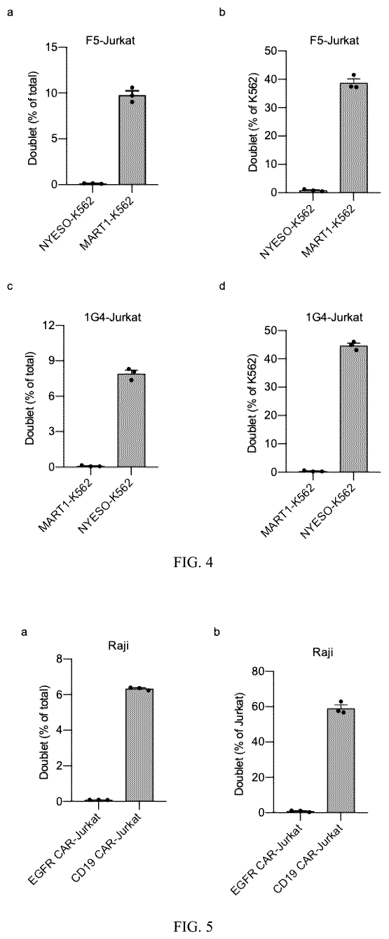

After co-culturing the cells that over-express TCR and SCT under the above conditions, further analyzed the fixed cell co-cultures by flow cytometry. In the paired group, 8%-9% of the total cells expressed both types of fluorescence (FIG. 4a, c) and approximately 40% of the cells in the SCT cell (CMFDA⁺) populations also expressed Violet fluorescence (FIG. 4b, d). The above results demonstrate that cell doublets forming can be used to identify the interaction between TCR and the target antigen thereof.

### Example 5. Identification of CAR-Specific Antigens by a Cell Adhesion Method

Genes encoding CD19 CAR or EGFR CAR and fusing to NGFR were constructed into an MSGV retroviral vector and transfected into 293T cells with a PEI transfection reagent for 48 hours and the virus was collected. Jurkat cells were infected with CAR for 48 hours and then sorted by flow cytometry to obtain cells highly expressing NGFR. CAR-Jurkat cells were labeled with Violet live cell tracking dye, and Raji cells (highly expressing CD19 itself, purchased from American Type Culture Collection (ATCC)) were labeled with CMFDA live cell tracking dye. These cells were then co-cultured at a cell ratio of Jurkat:Raji=1:20 at 37° C. for 30 mins and fixed with a fixative. The formation of Violet and CMFDA double-positive cell populations was analyzed by flow cytometry.

After co-culturing CAR-Jurkat cells (Violet⁺) with Raji cells (CMFDA⁺) under the above conditions, further analyzed the fixed cell co-cultures by flow cytometry. About 6.3% of the total cells expressed both types of fluorescence in the pair group (FIG. 5a) and approximately 60% of the cells in the CD19 CAR cell populations also expressed CMFDA fluorescence (FIG. 5b). The above results demonstrate that cell adhesion can be used to identify the interaction between CD19 CAR and target antigen CD19 thereof.

### Example 6. Identification of Viral Proteins and Receptors by a Cell Adhesion Method

Genes encoding COVID-19 spike protein (GenBank: QOP39313.1) and its receptor ACE2 (GenBank: AB046569.1) were constructed into a pcDNA vector (Addgene: V790-20) and instantaneously transfected into 293T cells with a PEI transfection reagent for 24 hours. 293T and spike-293T cells were labeled with CMFDA live cell tracking dye, and ACE2-293T cells were labeled with Violet live cell tracking dye. These cells were then co-cultured at a cell ratio of 1:5 at 37° C. for 30 mins and fixed with a fixative. The formation of Violet and CMFDA double-positive cell populations was analyzed by flow cytometry.

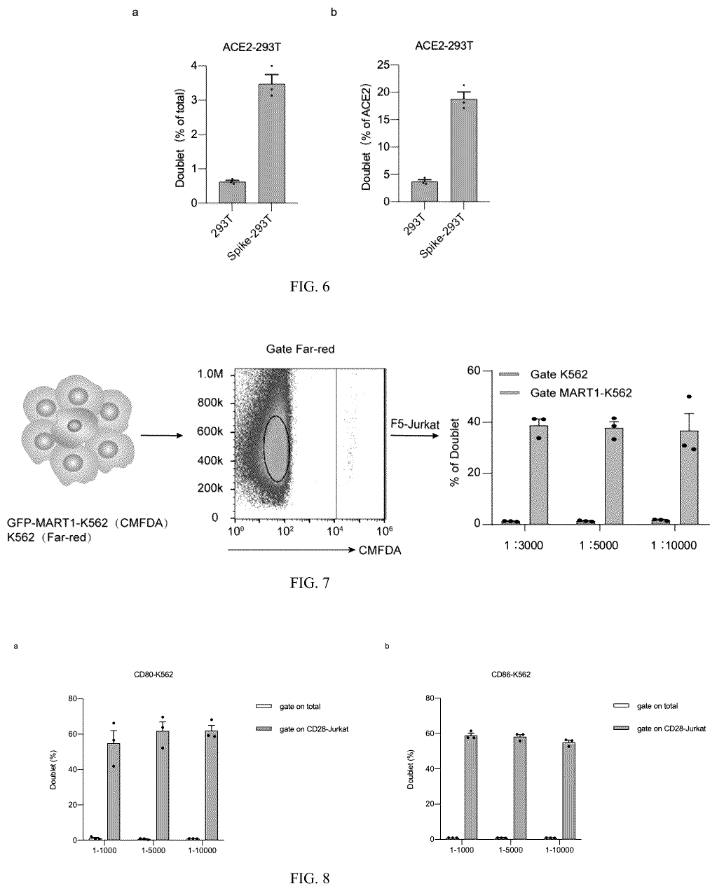

Further analyzed the fixed cell co-cultures by flow cytometry. About 3.5% of the total cells expressed both types of fluorescence in the pair group (FIG. 6a) and approximately 18.5% of the cells in the cell populations expressing ACE2 also expressed CMFDA fluorescence (FIG. 6b). The above results demonstrate that cell doublets forming can be used to identify the interaction between viral proteins and receptors thereof.

### Example 7. Separation of TCR-Specific Target Cells by a Cell Adhesion Method

To determine the sensitivity of cell adhesion, K562 cells overexpressing MART1 were diluted into K562 at a ratio of 1:3000, 1:5000, and 1:10000, respectively. The K562 cells overexpressing MART1 were labeled with CMFDA live cell tracking dye and then mixed with K562. The mixed cells were labeled with Far red live cell tracking dye. The mixed cells (Far red⁺, 2M) were mixed with F5-Jurkat cells (Violet+, 2M) (F5 TCR can specifically recognize MART1) at a ratio of 1:1. These cells were co-cultured at 37° C. for 30 mins and then fixed with a fixative. According to flow cytometry analysis, the proportion of adherent cells in K562-MART1 cells was about 40% (see FIG. 7), while the proportion of adherent cells in K562 cells was less than 5%. It demonstrates that SCT-K562 cells could be specifically binding to TCR which could be specifically recognized after diluting and the cell doublets forming has high sensitivity.

### Example 8. Specific Separation of Cells Expressing Target Proteins by a Cell Adhesion Method

To determine the sensitivity of cell adhesion, K562 cells overexpressing CD80 or CD86 were diluted into K562 at a ratio of 1:1000, 1:5000, and 1:10000, respectively. The K562 cells overexpressing CD80 or CD86 were labeled with CMFDA live cell tracking dye and then mixed with K562. The mixed cells were labeled with Far red live cell tracking dye. The mixed cells (Far red⁺, 2M) were mixed with CD28-Jurkat cells (Violet⁺, 2M) at a ratio of 1:1. These cells were co-cultured at 37° C. for 30 mins and then fixed with a fixative. Flow cytometry analysis shows that the proportion of adherent cells in K562-CD80 cells or K562-CD86 cells was about 60% (see FIG. 8), while the proportion of adherent cells in K562 cells was less than 3%. The above results demonstrate that CD80 or CD86-K562 cells could be specifically adhered to CD28-Jurkat which could be specifically recognized after diluting and the adhesion method has high sensitivity.

### Example 9. Screening for Target Cells of TCR from an SCT Library by a Cell Adhesion Method

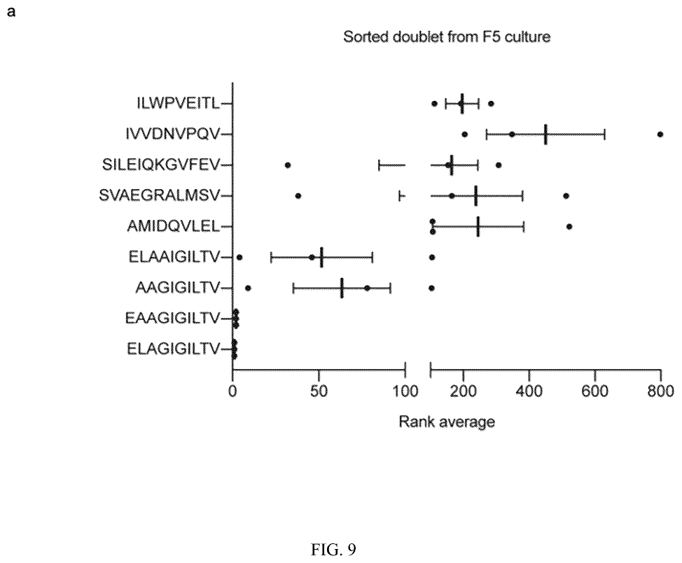

To further detect whether the adherence method can be applied to ligand library screening, a SCT cDNA library containing all known 12,055 HLA-A2 restricted T-cell epitopes from Immune Epitope Database (IEDB), including MART1 antigen peptides specifically recognized by F5 TCR, was constructed. The library was transduced into K562 cells, and the constructed A2 library-K562 cells (CMFDA⁺) were co-cultured with F5-Jurkat cells (Violet⁺) at a ratio of 1:1 (2M:2M) at 37° C. for 30 mins. Cells with both CMFDA⁺ and Violet⁺ were sorted by flow cytometry (see FIG. 9). DNA was extracted from the sorted cells and used as a template for amplification. The DNA was labeled with index primers and subjected to next-generation sequencing. The sequencing results were analyzed to determine whether the screened peptides were SCT paired with F5 TCR. As can be seen from FIG. 9, the screening platform has a high screening capacity for specifically recognized antigen peptides.

### Example 10. Assessment of the Titer of Bispecific Antibodies by a Cell Adhesion Method

Cells expressing a specific protein (e.g. CD3) and cells expressing another protein (e.g. CD19) on cell membranes were labeled with different fluorescent tracers, and then the two types of cells were co-cultured at a certain ratio in a system in which different bispecific antibodies were quantitatively added, respectively. After fixing with a fixative, the proportion of adherent cell populations expressing both types of fluorescence was detected by FACS. Different adhered cells were obtained by screening. The titer of the bispecific antibodies could be assessed by the level of cell doublets forming because the luminescent cells were binded together by the bispecific antibodies.

### Example 11. Screening for Interacting Proteins from a Membrane Protein Library by a Cell Adhesion Method

The cells transduced with the membrane protein cDNA library were labeled with different fluorescent tracers. The two different labeled cells were co-cultured at a certain cell ratio and then fixed with a fixative. Each pair of double fluorescent adherent cells was separately sorted into a 96-well plate by FACS. DNA was extracted respectively, amplified by PCR, and sequenced to obtain the sequence information on both interacting proteins. The method greatly shortens the time for screening the interacting proteins, improves work efficiency, and reveals the mechanism of action of different interacting proteins.

### Example 12. Improving the Accuracy of the Cell Adhesion Method for Screening Target Antigens by Combining an NFAT Promoter-Reporter Gene

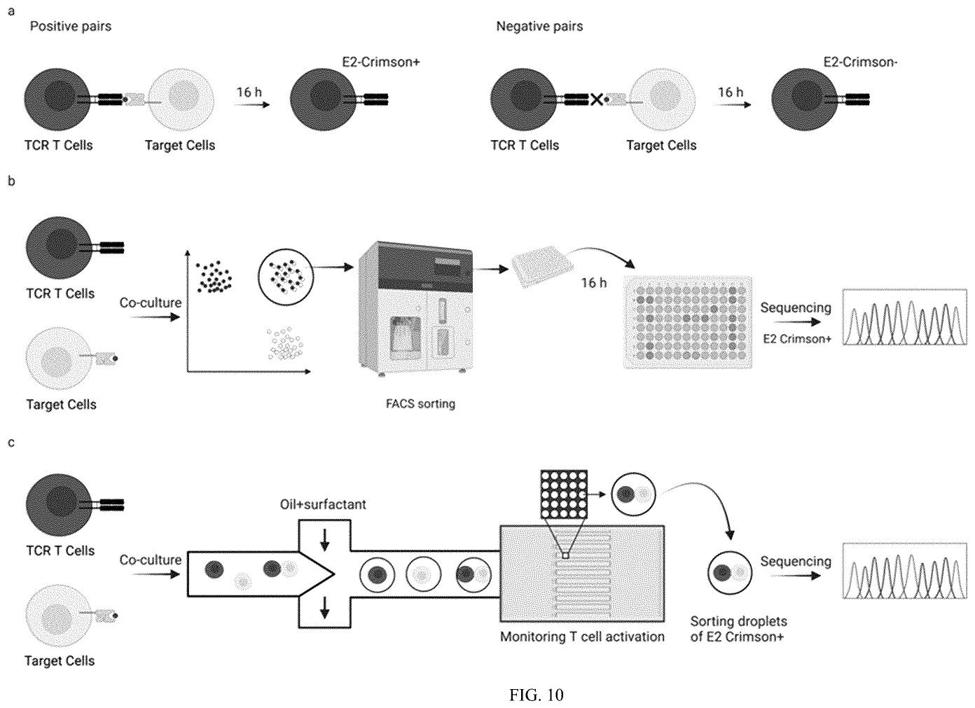

As previously described, the cell adhesion method can rapidly screen and separate cells expressing target antigens. To further improve the accuracy of the cell adhesion method for screening target antigens, an NFAT promoter-reporter gene E2-Crimson vector was constructed and transduced into cells expressing a specific TCR or TCR library by lentivirus infection. The above cells expressing the specific TCR or TCR library and transducing with the NFAT promoter-reporter gene and cells expressing a specific antigen or antigen library were labeled with different fluorescent tracers, and then the two cells were co-cultured at a certain ratio. Each pair of cell doublets was separately sorted into a 96-well plate by flow cytometry. After 16 hours of cultivation, the expression of E2-Crimson in each pair of adhered cells was detected by fluorescence microscopy (see FIG. 10), and if there is E2-Crimson expression, indicating that antigen-expressing cells in the pair of adhered cells can activate TCR-expressing cells. Furthermore, the paired cell doublets were lysed, and the gene sequences of TCR and antigen peptides therein were specifically amplified to obtain the gene information on the paired TCR-antigen. Likewise, the method can be further combined with different droplet microfluidic technologies to perform the above screening. The cell adhesion method combined with the NFAT promoter-reporter gene can more accurately identify TCR target antigens based on dual indicators that are cell-cell interactions to form specific adhesion bodies and signal stimulation of target antigens to activate the reporter gene expression, making it suitable for screening any type of antigen SCT cDNA library or TCR cDNA library.

It should be understood that the above examples are only used for verifying the feasibility of the present application, and should not be taken as limitations of the present application. According to the contents contained in the description of the present application, those skilled in the art would have been able to make conventional replacements for the types of cell surface markers in examples and use them to implement the technical solution of the present application without creative efforts.

1. A membrane protein interaction detecting platform, wherein the platform identifies interacting proteins by utilizing cell doublets generated by an interaction between proteins expressed on surfaces of cell membranes and the detecting platform comprises the following steps:
(1) co-culturing cells expressing different membrane proteins;
(2) fixing and screening cell doublets;
(3) obtaining sequences of interacting membrane proteins through sequence analysis.

2. The detecting platform of claim 1, wherein the step (1) further comprises labeling the cells with markers; preferably, fluorescent dyes are used for labeling, such as CMFDA, Violet, and Far red; more preferably, different cells are labeled with different kinds of dyes.

3. The detecting platform of claim 2, wherein the step (1) further comprises identifying the interaction between the membrane proteins through an expression of reporter genes, preferably, the reporter genes are used to identify an activation of TCR-expressing cells or T cells, and more preferably, an NFAT promoter is used to identify the activation of the TCR-expressing cells or the T cells.

4. The detecting platform of claim 1, wherein a method for screening the cell doublets in the step (2) comprises fluorescence-activated cell sorting (FACS) or droplet microfluidic technology; preferably, the screening is performed by FACS.

5. The detecting platform of claim 1, wherein the sequence analysis in the step (3) is performed by sequencing, and the sequencing is selected from Sanger sequencing or next-generation sequencing (NGS); preferably, the analysis is performed by next-generation sequencing, which comprises a synthetic sequencing platform, an Illumina/Solexa platform (e.g. HiSeq and MiSeq), a 454 pyrosequencing platform (Roche), or a SOLiD platform (Applied BioSystems); preferably, sequences are extracted and purified before sequencing, such as by PCR amplification.

6. The detecting platform of claim 1, wherein the step (1) further comprises a process of introducing expression vectors containing nucleic acid molecules encoding the membrane proteins into cells for expression, preferably, the expression vectors comprise any library expressing the membrane proteins; preferably a cDNA library; more preferably a SCT cDNA library.

7. The detecting platform of claim 3, wherein the interaction between the cell membrane proteins comprises binding between ligands and receptors, specific binding between TCRs and antigens, specific binding between CARs and antigens, or binding between viral proteins and receptors thereof.

8. The detecting platform of claim 1, wherein the step (1) further comprises expressing a nucleic acid sequence encoding membrane proteins in cells A by genetic engineering means, and co-incubating the cells A with identical or different cells B carrying the membrane proteins, resulting in contact.

9. The detecting platform of claim 1, wherein the membrane proteins are selected from transmembrane glycoproteins, G protein-coupled receptors, immunoglobulins, viral proteins, antigen recognition receptors, antibodies, antigenic determinants, cytokine receptors, low-density lipoprotein receptors, and any modified transmembrane proteins or polypeptides; preferably, the transmembrane glycoproteins are CD40 and CD40L; the immunoglobulins are CD28, CD80, and CD86; the viral protein is COVID-19 spike protein.

10. A method for separating and screening target cells using the detecting platform of claim 1, comprising: co-culturing the cells A expressing specific membrane proteins with target cells B to be separated and screened, the target cells B express proteins that can interact with the specific membrane proteins, fixing, separating, and screening the target cells B through an adhesion phenomenon between the cells A and B.

11. The method of claim 10, wherein the target cells comprise target cells that specifically bind to TCR, target cells that specifically bind to CAR, or cells that specifically bind to target proteins.

12. A method for screening interacting proteins from membrane proteins using the detecting platform of claim 1, wherein the method has one or more of the following uses:
(1) screening of T cell receptors (TCR) and target antigens thereof;
(2) screening of chimeric antigen receptors (CAR) and target antigens thereof;
(3) screening of antigens and antibodies or receptors thereof;
(4) screening of cytokines and receptors thereof.

13. The method of claim 12, wherein the target antigens of the TCR are selected from any HLA-SCT cDNA library, preferably a HLA-A2-SCT cDNA library.

14. A method for assessing a titer of a multispecific antibody using the detecting platform of claim 1, wherein the method comprises the following steps: labeling cells expressing specific proteins on cell membranes, co-culturing the labeled cells with the multispecific antibodies, fixing and detecting a proportion of labeled adherent cell populations to evaluate the titer of the multispecific antibody.

15. The method of claim 14, wherein detecting the proportion of the labeled adherent cell populations by FACS; preferably, the titer is evaluated by the proportion of the labeled adherent cell populations.

16. The method of claim 14, wherein the multispecific antibody is a bispecific antibody;
preferably, two different fluorescent tracers are used to label the cells, respectively.

17. The detecting platform of claim 1, wherein the detecting platform further comprises the following steps:
(1) co-culturing: mixing cells expressing different membrane proteins at an appropriate ratio, incubating under certain conditions;
(2) flow cytometry analyzing and sorting: fixing the cells after incubation to keep interacting cells in an adherent state, and sorting cells with both types of fluorescence simultaneously by a cell flow cytometry;
(3) sequencing and analyzing: amplifying screened cellular DNA by polymerase chain reaction (PCR) technology, and then performing next-generation sequencing to obtain membrane protein sequences that specifically adhere to known membrane proteins.
